(19) United States
(12) Patent Application Publication
(10) Pub. No.: US 2009/0299139 A1
(43) Pub. Date: Dec. 3, 2009

(54) ENDOSCOPE

(76) Inventor: Shinichi YAMAKAWA, Kanagawa (JP)

Correspondence Address: BIRCH STEWART KOLASCH & BIRCH, PO BOX 747, FALLS CHURCH, VA, 22040-0747, US

(21) Appl. No.: 12/472,115

(22) Filed: May 26, 2009

(30) Foreign Application Priority Data
May 27, 2008 (JP) 2008-138591
Aug. 12, 2008 (JP) 2008-207593

(51) Int. Cl. A61B 1/01

(52) U.S. Cl. 600/114

(57) ABSTRACT

An endoscope has a long slender insertion section to be inserted in a body cavity. The insertion section is composed by connecting a flexible portion to a distal portion having a CCD image sensor with a bending portion. Inserted through the insertion section are an air/water tube, a signal cable and other long slender contents. The air/water tube is enlarged in diameter in an area of the bending portion, and increases a filling rate of the contents in the bending portion.

### FIELD OF THE INVENTION

The present invention relates to an endoscope having a bending portion.

### BACKGROUND OF THE INVENTION

In the field of medicine, endoscopes are frequently used for diagnosis. A typical endoscope includes a long slender insertion section to be inserted into a body cavity of a patient, an operation section coupled to a base end of the insertion section, and a universal cord connected to a processing device and a light source device. The insertion section has a distal portion which incorporates an imaging unit having a solid state image sensor.

On a front end surface of the distal portion, there are provided one or more illumination windows for emitting illumination light to an internal body part, an image capturing window for receiving and directing image light of the body part into the imaging unit, and an air/water nozzle for spraying air or water over the image capturing window or the internal body part. The image light, introduced through the image capturing window and an objective lens behind it, is captured by the imaging unit, and converted into an image signal. This image signal is delivered to the processing device by way of a signal cable running through the insertion section, the operation section and the universal cord. Along with the signal cable, a plurality of long slender contents, such as an optical fiber cable for carrying the illumination light to the illumination window and an air/water tube for delivering air or water to the air/water nozzle, extend through the insertion section, the operation section and the universal cord.

The distal portion is attached to a bending portion which is connected to a flexible portion. The bending portion is composed of a plurality (for example, sixteen) of annular joint pieces connected in series to one another. Adjacent joint pieces are pivotally joined together. Inside the joint pieces, there are two pairs of operation wires: one for vertical turn and the other for horizontal turn. Pushing and pulling these operation wires leads the joint pieces to turn, and thereby the bending portion as whole to bend in the vertical or horizontal direction.

An angle knob to push and pull the operation wires is provided on the operation section. In response to the operation of the angle knob, the bending portion bends up and down or left and right to point the distal portion in a desired direction. This bending action leads the contents, such as cables and tubes, to slide in their longitudinal direction inside the bending portion. To facilitate sliding of the contents, a gap is created inside the bending portion. On the other hand, the bending action of the bending portion produces a force that pushes the contents in a radial direction perpendicular to the longitudinal direction. Because of the gap inside the bending portion, the contents move also in the radial direction. In this situation, however, the contents would get rubbed, twisted and tangled as they move further in the radial direction, and disturb the bending action of the bending portion. Even worse, the contents would possibly be damaged.

In view of this drawback, Japanese Patent Laid-open Publication No. 2001-137178 discloses an endoscope which has a metallic coil placed in the bending portion. This coil narrows the gap inside the bending portion to restrict the radial movement of the contents. Also, Japanese Patent No. 3181707 discloses an endoscope having an elastic tube inserted into the bending portion to fill the gap therein.

Further, there is disclosed an endoscope whose contents are bundled and fixed to restrict their radial movement (see, for example, Japanese Patent Laid-open Publications No. 05-303044, No. 2006-025985, No. 2007-296141 and Japanese Patent No. 2842616).

However, the endoscopes of the Publication No. 2001-1317178 and the Patent No. 3181707 require a separate movement restricting member, such as the coil or the elastic tube. In addition, filling the gap with movement restricting member leads to increase a filling rate of the contents in the bending portion, and impedes the contents to slide in the longitudinal direction. Structurally, the inner diameter of the insertion section is narrow at a connection point between the bending portion and the flexible portion, and the movement restricting member significantly increases the filling rate in this connection point, impeding the contents to slide in their longitudinal direction.

The increase in the filling rate at the connection point may be avoided by shortening the movement restricting member to extend only across the bending portion. With this configuration, however, the terminal ends of the movement restricting member may possibly be caught between the joint pieces, and disturb the bending action. In some situations, the movement restricting member may break or jut out from between the joint pieces.

Even in the case of bundling and fixing the contents, the longitudinal slide of the contents is also impeded, and such impediment disturbs the bending action, and even worse, may break the contents.

### SUMMARY OF THE INVENTION

In view of the foregoing, it is an object of the present invention to provide at low cost an endoscope for capable of preventing damage to contents of an insertion section, without disturbing bending action of the insertion section.

In order to achieve the above and other objects, an endoscope according to the present invention includes a long slender insertion section having a bending portion and being inserted into a body cavity, and a long slender content inserted through the insertion section. This content is enlarged in diameter in an area of the bending portion, and restricted from moving in a direction perpendicular to its longitudinal direction.

In another preferred embodiment, the endoscope includes a tube for covering and enlarging the content in the area of the bending portion. This tube has higher thermal conductivity than the content. It is preferred to embed a metal mesh in the tube.

In yet another preferred embodiment, the content is enlarged in diameter at a position of maximum curvature in the area of the bending portion upon bending.

An endoscope according to the present invention includes a long slender insertion section having a bending portion and being inserted into a body cavity, a plurality of long slender contents inserted through the insertion section, and a tube for holding at least two of the contents while allowing the contents to slide in their longitudinal direction.

It is preferred that a condition D1>D2 is satisfied, wherein D1 represents an inner diameter of the tube, and D2 represents a diameter of a circumscribed circle of the two contents in contact with each other. More preferably, a condition D1>D2×1.118 is satisfied.

It is preferred that the tube extends at least between a leading end and a position of maximum curvature of the bending portion, and at most throughout the entire length of the bending portion. Preferably, the tube has a complete circular cross section.

The tube is preferably made of fluorine resin, polyurethane resin or elastomeric resin. It is also preferred to embed a metal mesh in the tube.

These contents are an air/water tube for delivering air and water, a signal cable for transmitting an image signal produced from an imaging unit, an optical fiber cable for carrying illumination light from a light source device to an illumination window, and a forceps tube for inserting a medical instrument.

According to the present invention, the filling rate of the contents is increased in the bending portion to restrict the movement of the contents in the direction perpendicular to their longitudinal direction. Prevented from rubbing, twisting and tangling, the contents are protected from damage. Since the filling rate is not increased at the connection point between the bending portion and the flexible portion, the contents are able to slide easily in the insertion section, and the bending action of the insertion section is not disturbed.

In the case of bundling the contents, the filling rate in the bending portion is increased by the tube to hold the contents. The increased filling rate regulates the movement of the contents in the direction perpendicular to their longitudinal direction, serving to protect the contents from damage. In addition, the tube has the inner diameter larger than the diameter of the circumscribed circle of the contents, and allows the contents to slide in their longitudinal direction. Being shorter than or equal to the entire length of the bending portion, this tube does not increase the filling rate at the connection point between the bending portion and the flexible portion. Therefore, the bending action of the insertion section is not disturbed.

### BRIEF DESCRIPTION OF THE DRAWINGS

The above objects and advantages of the present invention will become more apparent from the following detailed description when read in connection with the accompanying drawings, in which:

FIG. 1 is an external view of an endoscope according to the present invention;

FIG. 2 is a cross sectional view of a bending portion;

FIG. 3 is an axial cross sectional view of the bending portion showing an air/water tube;

FIG. 4 is an axial cross sectional view of a bending portion having another air/water tube;

FIG. 5 is an axial cross sectional view of a bending portion having yet another air/water tube;

FIG. 6 is an axial cross sectional view of a signal cable with a partly enlarged diameter;

FIG. 7 is a cross sectional view of a bending portion having a tube for holding an air tube and a water tube;

FIG. 8 is an axial cross sectional view of the bending portion showing the tube inserted therein; and

FIG. 9 is a table for deformation ratios of the tube in bending of the bending portion.

### DETAILED DESCRIPTION OF THE PREFERRED EMBODIMENTS

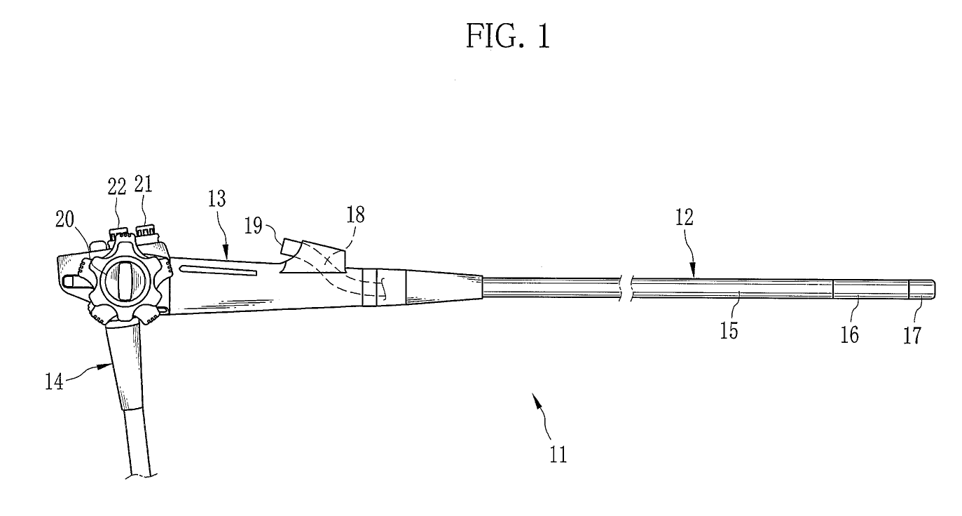

Referring to FIG. 1, an endoscope 11 includes a long slender and round bar-shaped insertion section 12 to be inserted into a body cavity, an operation section 13 as a grip of the endoscope 11 and for operating the insertion section 12, and a universal cord 14 extending from the operation section 13. The insertion section 12 is composed of a flexible portion 15 at a base end, a bending portion 16 in the middle, and a distal portion 17 at a leading end.

The flexible portion 15 is slightly bendable, and occupies most of the insertion section 12. The bending portion 16 bends up and down or left and right in accordance with the operation of the operation section 13, and turns the distal portion 17 around.

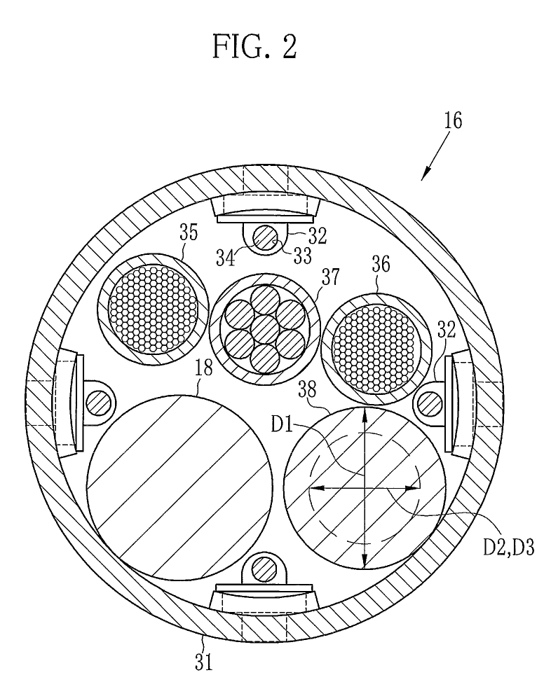

On a front end surface of the distal portion 17, there are provided two illumination windows, an image capturing window, an air/water nozzle, and a forceps channel opening (all not shown). Illumination light from a light source device (not shown) connected to the universal cord 14 is delivered on the optical fiber cables 35, 36 (see, FIG. 2) to the illumination window, and emitted from the illumination window to an internal body part. These optical fiber cables 35, 36 extend through the insertion section 12, the operation section 13 and the universal cord 14.

Behind the image capturing window, as illustrated in U.S. Pat. No. 7,201,717, there are arranged an objective lens and an imaging unit having a CCD or another type of image sensor. The imaging unit is connected to a signal cable 37 (see, FIG. 2) that extends through the insertion section 12, the operation section 13 and the universal cord 14. The imaging unit captures an image of the internal body part through the image capturing window and the objective lens, and converts the image into an image signal. This image signal is transmitted through the signal cable 37 to a processing device (not shown) connected to the universal cord 14. The image signal in the processing unit is applied to a predetermined image processing, and displayed as an endoscopic image on a monitor (not shown).

The air/water nozzle is connected to an air/water tube 38 (see, FIG. 2) that extends through the insertion section 12, the operation section 13 and the universal cord 14. Cleaning water or air is fed into the air/water tube 38, and sprayed over the image capturing window or an internal body part from the air/water nozzle.

The operation section 13 includes a forceps insertion port 19, an angle knob 20, an air/water feed button 21 and a suction button 22. The forceps insertion port 19 is coupled to one end of the forceps tube 18. A medical instrument, such as a forceps or a syringe needle is inserted from the forceps insertion port 19, and projects into a body cavity from the forceps channel opening on the distal portion 17. The angle knob 20 is operated to change a direction and an angle of the bending portion 16. By rotating the angle knob 20, operation wires 34 (see, FIG. 2) are pushed or pulled to bend the bending portion 16.

The air/water feed button 21 is pressed to feed the cleaning water or air into the air/water tube 38. The suction button 22 is pressed to suck liquid or a tissue inside the body cavity into the forceps tube 18.

As shown in FIG. 2, the bending portion 16 is composed of a plurality (for example, sixteen) of tubular joint pieces 31. These joint pieces 31 are coupled to one another with connection pins 32. Each connection pin 32 has a guide hole 33 to fit onto the operation wire 34. The joint pieces 31 enclose the forceps tube 18, the optical fiber cables 35, 36, the signal cable 37 and the air/water tube 38.

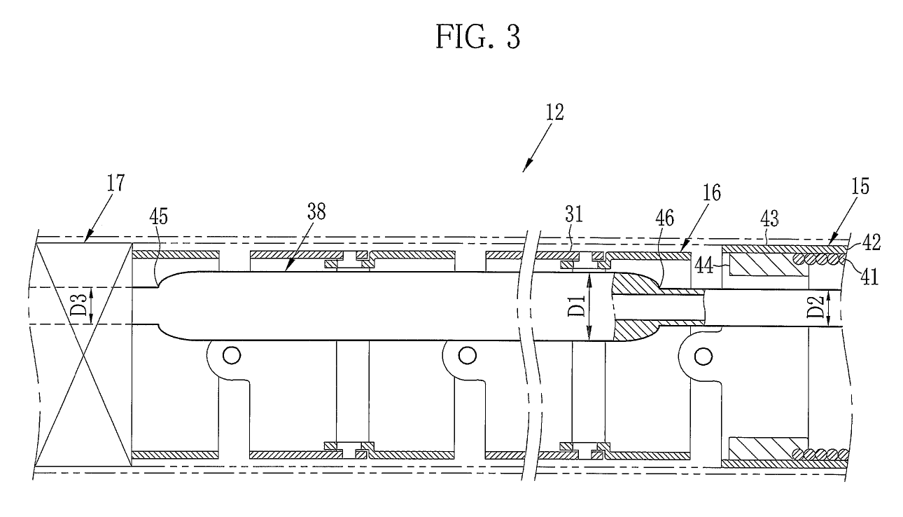

As shown in FIG. 3, the flexible portion 15 is composed of a coil 41 and an outer tube 42 for covering the coil 41. The coil 41 is fixed to the bending portion 16 with a retainer 44 at a connection point 43.

The air/water tube 38 is enlarged in diameter in the bending portion 16. Specifically, the air/water tube 38 has an outer diameter D1 in the bending portion 16 enlarged with respect to its outer diameter D2 in the flexible portion 15 and its outer diameter D3 in the distal portion 17 (D1>D2, D1>D3). The outer diameter D2 is of the same size as the outer diameter D3 (D2=D3). Diameter changing points 45, 46 between the diameters D1 and D3 and between the diameters D1 and D2 are both located in the bending portion 16. In FIG. 3, the other contents than the air/water tube 38 are omitted for the sake of simplicity.

In this manner, by enlarging the outer diameter D1 with respect to the outer diameters D2, D3, a filling rate of the contents is increased in the bending portion 16. The increased filling rate restricts the contents from moving in a radial direction (perpendicular to their longitudinal direction) inside the bending portion 16. Explained with FIG. 2, the enlarged outer diameter narrows a gap at lower right from the air/water tube 38, and prevents other contents, such as the optical fiber cable 36 above the air/water tube 38, from going beyond the right-side connection pin 32 and moving down into the area below upon bending of the bending portion 16. This prevents rubbing, twisting and tangling of the contents, protecting thereby the contents from damage in bending of the bending portion 16. Even so, however, the gap in the bending portion 16 is not completely filled, and the contents are still able to slide in their longitudinal direction. Therefore, the bending action of the bending portion 16 is not disturbed.

Requiring no dedicated part, the present invention does not cause increase in production cost and in the number of process steps. In the event that the bending portion 16 has an even smaller diameter, the present invention does not hinder such downsizing.

As shown in FIG. 3, the diameter changing point 46 between the diameter D1 and the diameter D2 is located in the bending portion 16, and the diameter of the air/water tube 38 is not increased at the connection point 43. This prevents significant increase in the filling rate at the connection point 43 where the inner diameter is relatively small due to the presence of the retainer 44. The contents are therefore able to slide in the longitudinal direction, and the bending action of the bending portion 16 is not disturbed.

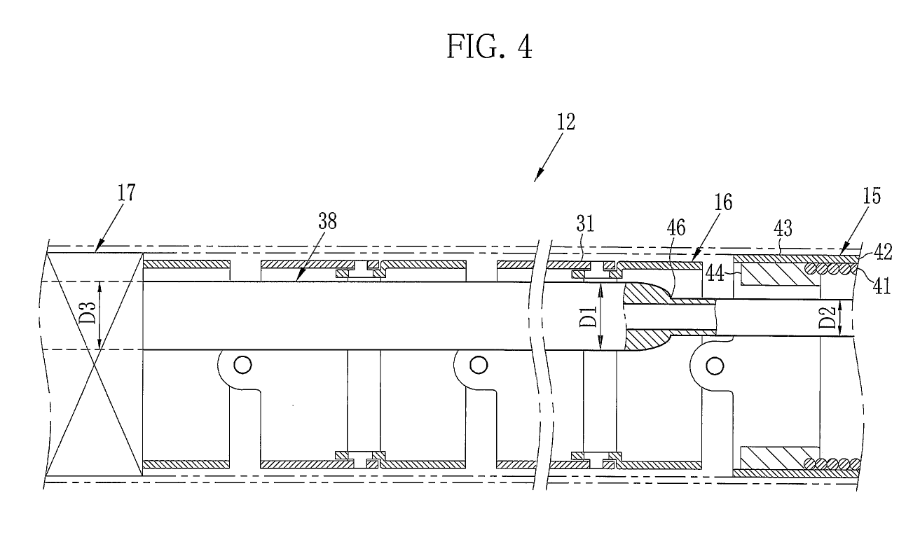

Since it is only necessary to increase the filling rate in the bending portion 16 without raising the filling rate in the connection point 43, the outer diameter D3 of the air/water tube 38 may be of the same size as the outer diameter D1 (D1=D3), as shown in FIG. 4.

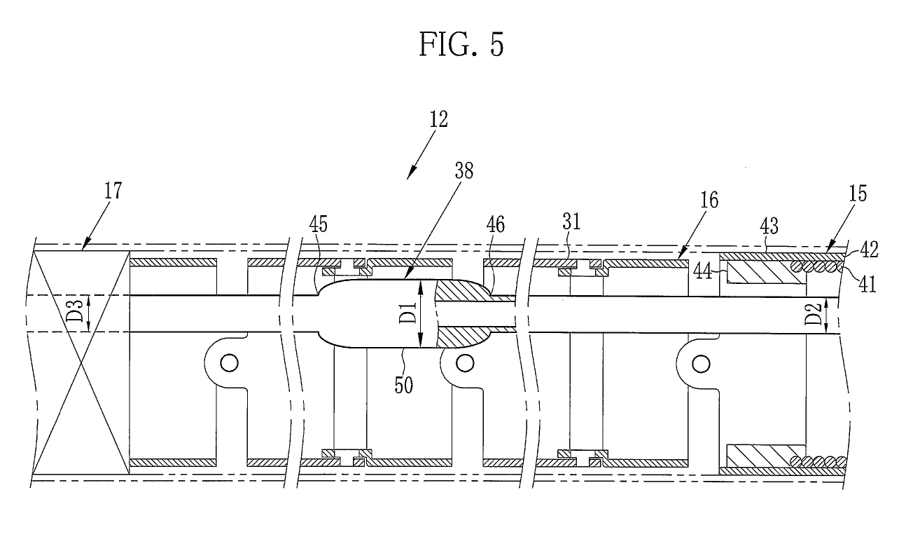

In the above embodiment, the air/water tube 38 extends with the outer diameter D1 almost throughout the bending portion 16. However, as shown in FIG. 5, the diameter of the air/water tube 38 may be increased only at a position 50 of maximum curvature (namely, minimum radius of curvature) in bending of the bending portion 16. Additionally, the outer diameter of the air/water tube 38 may be increased gradually or continuously, so that it peaks at the position of maximum curvature.

Although the above embodiment is directed to the air/water tube 38 to increase the filling rate in the bending portion 16, it is possible to enlarge the outer diameter of any one or all of the signal cable 37, the optical fiber cables 35, 36 and the forceps tube 18, instead of or in addition to the air/water tube 38 in the bending portion 16.

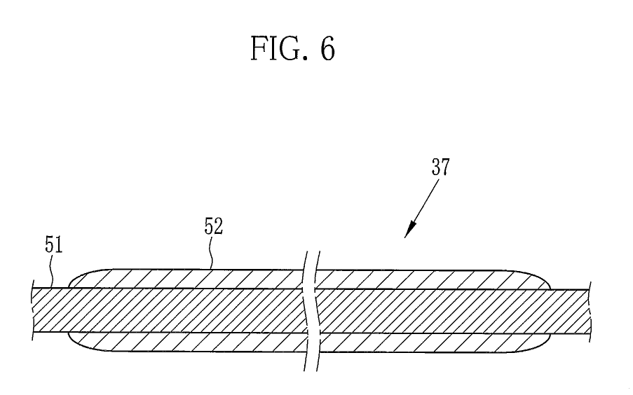

If the signal cable 37 is to be enlarged, it is preferred to cover the outer periphery thereof with a high thermal conductivity material. This covering promotes heat dissipation from the image sensor (CCD), as well as increasing the filling rate in the bending portion 16. For example, the signal cable 37 in FIG. 6 is composed of a cable body 51 and a tube 52 wrapped around the cable body 51. The tube 52 is made of a material with higher thermal conductivity than the cable body 51. To improve the heat dissipation performance, the tube 52 contains an embedded metal mesh or the like. This signal cable 37 is easy to manufacture as it only requires covering the cable body 51 with the tube 52. With higher thermal conductivity than the cable body 51, the tube 52 serves to promote dissipation of the heat generated from the optical fiber cables 35, 36 nearby the signal cable 37. Similarly, the other contents, such as the air/water tube 38 may be covered with this type of tube.

Additionally, a single large-diameter tube may be used to bundle several contents as described below. Hereafter, the elements similar to those in the above embodiment are designated by the same reference numerals, and the detailed explanations thereof are omitted.

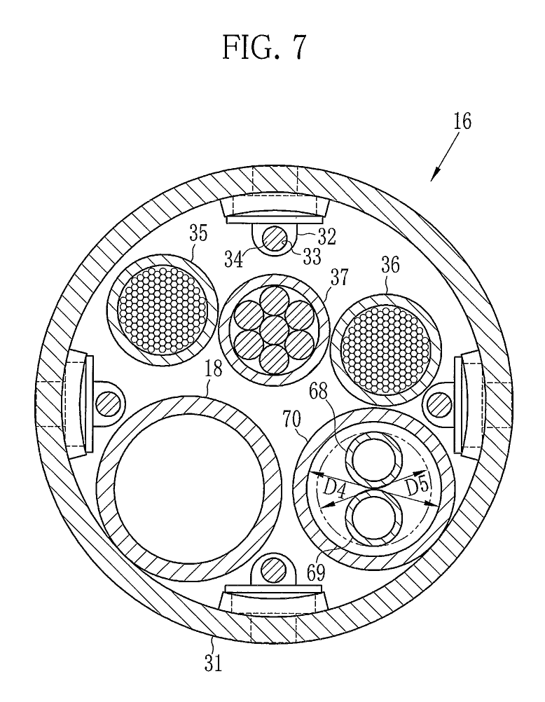

As shown in FIG. 7, this embodiment uses an air tube 68 and a water tube 69, in place of the air/water tube 38. These air and water tubes 68, 69 run through the insertion section 12, the operation section 13 and the universal cord 14, and are connected on their tips to the air/water nozzle. It may also be possible to provide an air nozzle and a water nozzle in place of the air/water nozzle, and to connect the air tube 68 and the water tube 69 to these nozzles separately.

The air tube 68 and the water tube 69 are bundled together with a tube 70 having a complete circular cross-section. The tube 70 has an inner diameter D4 slightly larger than a diameter D5 of a circumscribed circle (illustrated by a chain double-dashed line) of the air and water tubes 68, 69 in contact with each other. By determining the inner diameter D4 to meet the condition D1>D2, a gap is created around the air tube 68 and the water tube 69. This gap allows the air tube 68 and the water tube 69 to slide in their longitudinal direction in the tube 70. Preferably, the inner diameter D4 is determined to satisfy the condition D4>D5×1.118, so that the gap is maintained in the tube 70 when the tube 70 is deformed upon bending of the bending portion 16.

The tube 70 is made of an elastic material such as, preferably, fluorine resin, polyurethane resin or elastomeric resin. For more intensity, a metal mesh may be embedded in the tube 70, or a porous material may be used to reinforce the tube 70.

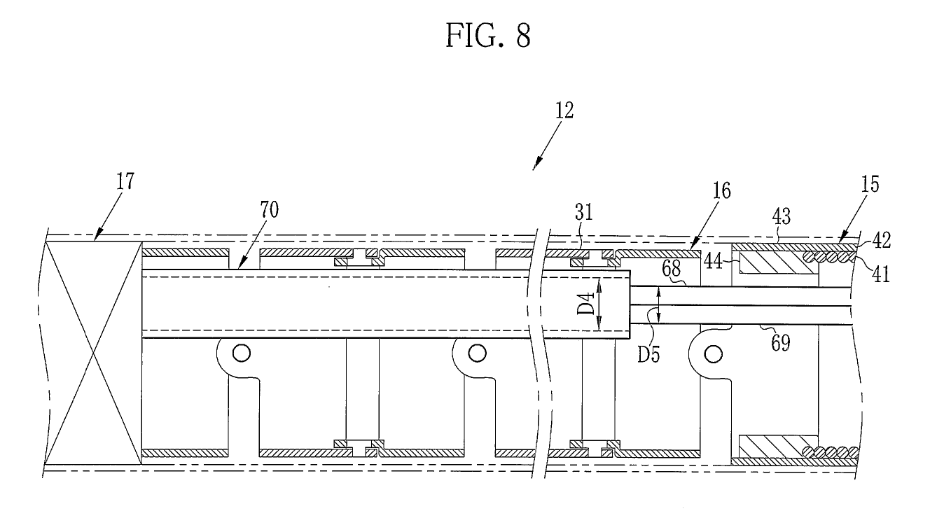

As shown in FIG. 8, the tube 70 is fixed on one end to the distal portion 17. The tube 70 extends close to the connection point 43 between the bending portion 16 and the flexible portion 15. In FIG. 8, the other contents are omitted for the sake of simplicity.

The large-diameter tube 70 around the air and water tubes 68, 69 increases the filling rate in the bending portion 16. The increased filling rate prevents the contents, such as the forceps tube 18, the optical fiber cables 35, 36, the signal cable 37 and the tube 70, from going beyond the connection pins 32 and getting twisted and tangled.

Since the gap is created in the tube 70 because of the inner diameter D4 of the tube 70 which is made slightly larger than the diameter D5 of the circumscribed circle of the air and water tubes 68, 69, the air tube 68 and the water tube 69 can slide in the longitudinal direction. Therefore, the bending action of the bending portion 16 is not disturbed. Additionally, the movement of the air tube 68 and the water tube 69 in the radial direction is restricted within the tube 70, and twisting and tangling of the air and water tubes 68, 69 are prevented. This facilitates the bending action, and serves to protect the contents from damage due to twisting and tangling. Because of the complete circular cross section, the tube 70 has no bending anisotropy, and provides a certain level of bending rigidity in all directions. Therefore, the tube 70 is hardly damaged by the bending action of the bending portion 16.

### EXAMPLE 1

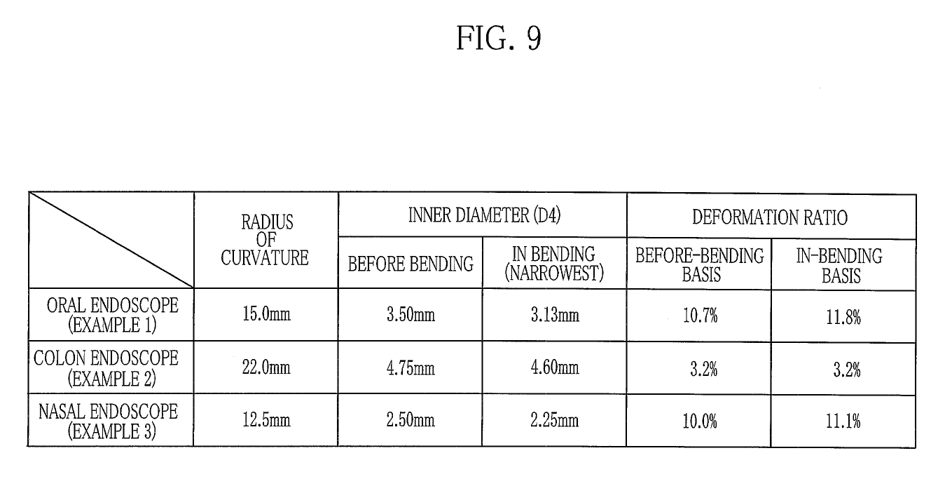

Next, examples of the present invention are described. In the following first to third examples, a deformation ratio of the tube 70 was measured in bending of the bending portion 16, using an oral endoscope, a colon endoscope and a nasal endoscope. As shown in a table of FIG. 9, the bending portion 16 of the oral endoscope 11 measured the minimum radius of curvature of 15.0 mm when it was bent until the front surface of the distal portion 17 turned by 180°. Before bending of the bending portion 16, the inner diameter D4 of the tube 70 was 3.50 mm. In bending, by contrast, the inner diameter D4 was reduced to 3.13 mm at the narrowest. The deformation ratio of the inner diameter D4 reached substantially 10.7% drop on the basis of the before-bending dimension ((D4 in bending (narrowest)−D4 before bending)/D4 before bending). On the basis of the in-bending dimension ((D4 in bending (narrowest)−D4 before bending)/D4 in bending (narrowest)), the deformation ratio of the inner diameter D4 reached substantially 11.8% drop.

### EXAMPLE 2

The bending portion 16 of the colon endoscope 11 measured the minimum radius of curvature of 22.0 mm when it was bent in the same manner as Example 1. Before bending of the bending portion 16, the inner diameter D4 of the tube 70 was 4.75 mm. In bending, by contrast, the inner diameter D4 was reduced to 4.60 mm at the narrowest point. The deformation ratio of the inner diameter D4 reached substantially 3.2% drop on the basis of the before-bending dimension, and also substantially 3.2% drop on the basis of the in-bending dimension.

### EXAMPLE 3

The bending portion 16 of the nasal endoscope 11 measured the minimum radius of curvature of 12.5 mm when it was bent in the same manner as Example 1. Before bending of the bending portion 16, the inner diameter D4 of the tube 70 was 2.50 mm. In bending, by contrast, the inner diameter D4 was reduced to 2.25 mm at the narrowest point. The deformation ratio of the inner diameter D4 reached substantially 10.0% drop on the basis of the before-bending dimension, and substantially 11.1% drop on the basis of the in-bending dimension.

To allow the air tube 68 and the water tube 69 to slide in the longitudinal direction, the gap needs to be maintained in the tube 70 even in bending of the bending portion 16 (during deformation of the tube 70). If the diameter D5 of the circumscribed circle of the air and water tubes 68, 69 is smaller than the inner diameter D4 (at the narrowest point) of the tube 70 in bending, the air tube 68 and the water tube 69 would slide easily in the longitudinal direction (A-grade slidability). If the diameter D5 is substantially the same as the inner diameter D4 of the tube 70 during bending, the air tube 68 and the water tube 69 would be able to slide in the longitudinal direction (B-grade slidability). In contrast, if the diameter D5 is smaller than the inner diameter D4 of the tube 70 in bending, the air tube 68 and the water tube 69 would not slide in the longitudinal direction (C-grade slidability).

In Example 1, the slidability is “C” under the condition D4<D5×1.118. The slidability is “B” under the condition D4=D5×1.118, while it is “A” under the condition D4>D5×1.118.

In Example 2, the slidability is “C” under the condition D4<D5×1.032. The slidability is “B” under the condition D4=D5×1.032, while it is “A” under the condition D4>D5×1.032.

In Example 3, the slidability is “C” under the condition D4<D5×1.111. The slidability is “B” under the condition D4=D5×1.111, while it is “A” under the condition D4>D5×1.111.

These examples prove that the A-grade slidability is achieved to all the oral, colon, and nasal endoscopes 11 under the condition D4>D5×1.118.

Although the gap is created in the tube 70 in this embodiment, a different configuration may be used insofar as it ensures the appropriate slidability of the air tube 68 and the water tube 69 in the longitudinal direction. For example, the air tube 68 and the water tube 69 may be made of a material with a low friction coefficient, and they are tightly inserted in the tube without a gap.

It is possible to adjust the tube 70 to a desired length within the range of at least between the leading end of the bending portion 16 and the position of maximum curvature of the bending portion 16 (for example, the midpoint in the length of the bending portion 16), and at most the entire length of the bending portion 16.

While the air tube 68 and the water tube 69 are bundled with the tube 70 in the above embodiment, it is possible to bundle at least two of the forceps tube 18, the optical fiber cables 35, 36, the signal cable 37, the air/water tube with a tube.

Although the above embodiments are directed to medical endoscopes, the present invention is preferably applicable to industrial endoscopes.

Although the present invention has been fully described by the way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An endoscope comprising:
a long slender insertion section to be inserted into a body cavity, and having a bending portion; and
a long slender content inserted through said insertion section, and being enlarged in diameter in an area of said bending portion.

2. The endoscope of claim 1, further comprising a tube for covering and enlarging said content in said area of said bending portion.

3. The endoscope of claim 2, wherein said tube has higher thermal conductivity than said content.

4. The endoscope of claim 3, wherein a metal mesh is embedded in said tube.

5. The endoscope of claim 1, wherein the diameter of said content is largest at a position of maximum curvature in said area upon bending of said bending portion.

6. The endoscope of claim 1, wherein said content is one of an air/water tube for delivering air and water, a signal cable for transmitting an image signal produced from an imaging unit, an optical fiber cable for carrying illumination light from a light source device to an illumination window, and a forceps tube for inserting a medical instrument.

7. An endoscope comprising:
a long slender insertion section to be inserted into a body cavity, and having a bending portion;
a plurality of long slender contents inserted through said insertion section; and
a tube for holding at least two of said contents while allowing said contents to slide in their longitudinal direction.

8. The endoscope of claim 7, wherein said tube satisfies a condition of D1>D2,
wherein D1 represents an inner diameter of said tube, and D2 represents a diameter of a circumscribed circle of said two contents in contact with each other.

9. The endoscope of claim 8, wherein said tube further satisfies a condition of D1>D2'1.118.

10. The endoscope of claim 7, wherein said tube extends at least between a leading end of said bending portion and a position of maximum curvature of said bending portion, and at most between said leading end and a base end of said bending portion.

11. The endoscope of claim 7, wherein said tube has a complete circular cross section.

12. The endoscope of claim 7, wherein said tube is made of fluorine resin, polyurethane resin or elastomeric resin.

13. The endoscope of claim 7, wherein a metal mesh is embedded in said tube.

14. The endoscope of claim 7, wherein said contents are an air/water tube for delivering air and water, a signal cable for transmitting an image signal produced from an imaging unit, an optical fiber cable for carrying illumination light from a light source device to an illumination window, and a forceps tube for inserting a medical instrument.
